(19) United States
(12) Patent Application Publication
(10) Pub. No.: US 2006/0056986 A1
(43) Pub. Date: Mar. 16, 2006

(54) Multi-cylinder compressor

(75) Inventors: Jong Won Seok, Suwon-si (KR); Valeri Lenchine, Suwon -si (KR); Jin Woo Lee, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 11/128,154

(22) Filed: May 13, 2005

(30) Foreign Application Priority Data
Sep. 15, 2004 (KR) 2004-73808

(51) Int. Cl. F04B 1/04; A61M 1/00; F04B 27/04

(52) U.S. Cl. 417/273; 417/437

(57) ABSTRACT

A multi-cylinder compressor is designed to have excellent noise and pulsation reduction and also be easy to manufacture. The multi-cylinder compressor includes first and second compressing compartments partitioned from each other to perform compression of gas, respectively, first and second mufflers equipped to discharge openings of the first and second compressing compartments, respectively, and first and second discharge flow paths extended a predetermined length from the first and second mufflers so as to reduce noise and pulsation while guiding discharge of compressed gas.

### CROSS-REFERENCE TO RELATED APPLICATION

This application claims priority from Korean Patent Application No. 2004-73808, filed on Sep. 15, 2004 in the Korean Intellectual Property Office, the disclosure of which is incorporated herein by reference.

### BACKGROUND OF THE INVENTION

1. Field of the Invention

The present invention relates to a multi-cylinder compressor and, more particularly, to a multi-cylinder compressor designed to allow easy manufacturing and to enhance noise and pulsation reduction of discharge gas in the compressor.

2. Description of the Related Art

A multi-cylinder rotary type compressor disclosed in Japanese Patent Laid-open Publication No. 2000-320479 (Laid-open Date: Nov. 21, 2000) comprises a first compressing compartment defined at an upper portion, and a second compressing compartment defined at a lower portion, which can be partitioned from each other upon rotation of a motor, thereby allowing a refrigerant gas to be compressed in the first and second compressing compartments. The compressor further comprises a first muffler equipped at an upper side of the first compressing compartment, and a second muffler equipped at a lower side of the second compressing compartment in order to reduce noise and pulsation caused by discharge gas from the first and second compressing compartments.

Additionally, the compressor has a gas pathway vertically defined through a first cylinder constituting the first compressing compartment, a second cylinder constituting the second compressing compartment, and a partition plate disposed between the first and second cylinders, such that the first muffler communicates with the second muffler via the gas pathway. The middle plate is provided with a Helmholtz resonator communicated with the gas pathway. The first muffler has a discharge opening opened such that gas discharged from the first compressing compartment into the first muffler, and gas discharged from the second muffler into the first muffler through the gas pathway can be discharged into a closed container.

Such a construction allows noise to be reduced by virtue of reflection and interference of the noise and pulsation of the discharge gas within the second muffler and the gas pathway while the gas discharged from the second compressing compartment into the second muffler flows to the first muffler through the gas pathway. In particular, as the discharged gas passes through the gas pathway, the noise and pulsation can be further reduced by virtue of operation of the Helmholtz resonator. Moreover, the gas discharged from the first compressing compartment at the upper portion of the compressor is injected into the first muffler, and discharged to the outside through the discharge opening after the noise and pulsation is reduced.

Such a noise and pulsation reduction device of the multi-cylinder compressor can reduce the noise and pulsation of the gas discharged from the second compressing compartment by virtue of operations of the second muffler, the gas pathway, and the Helmholtz resonator. However, as for the gas discharged from the first compressing compartment into the first muffler, because it is discharged through the discharge opening of the first muffler directly after passing through the first muffler, noise and pulsation reduction of the discharge gas is not satisfactory. In particular, as for the discharge opening of the conventional first muffler, since the discharge opening not only is directly communicated with the interior of the closed container, but also has a relatively large size, thereby providing a minute influence on the reduction in noise and pulsation transferred through the discharge opening (that is, it does not serve as a soundproof structure), the noise and pulsation reduction of the discharge gas is not satisfactory.

Moreover, in such a noise and pulsation reduction device, interaction between the noise and pulsation transferred into the first muffler through the second muffler and the gas pathway and the noise and pulsation transferred from the first compressing compartment into the first muffler occurs within the first muffler, thereby amplifying noise and pulsation within a specific frequency band, which can be easily transferred into the closed container through the discharge opening provided at the upper portion of the first muffler, leading to unsatisfactory noise and pulsation reduction of the discharge gas.

Moreover, although the noise and pulsation reduction device is realized in order to enhance the noise reduction by means of the Helmholtz resonator, which is provided through the partition plate to communicate with the gas pathway, a complicated process for drilling a cavity and a neck through the partition plate is required in order to prepare the Helmholtz resonator, thereby complicating the manufacturing of the compressor.

### SUMMARY OF THE INVENTION

The present invention has been made in view of the above and other problems, and an aspect of the present invention is to provide a multi-cylinder compressor, designed to allow easy manufacturing, and have excellent noise and pulsation reduction effect.

Additional aspects and/or advantages of the invention will be set forth in part in the description which follows and, in part, will be apparent from the description, or may be learned by practice of the invention.

In accordance with one aspect, a multi-cylinder compressor is provided comprising: first and second compressing compartments partitioned from each other to perform compression of gas, respectively; first and second mufflers equipped to discharge openings of the first and second compressing compartments, respectively; and first and second discharge flow paths extended a predetermined length from the first and second mufflers so as to reduce noise and pulsation while guiding discharge of compressed gas.

Both the first and second discharge flow paths may have a length larger than a width of a cross section thereof.

The multi-cylinder compressor may further comprise: first and second cylinder bodies constituting the first and second compressing compartments, respectively; first and second compressing devices disposed within the first and second compressing compartments, respectively; a rotational shaft penetrating through the first and second compressing compartments to drive the first and second compressing devices; a partition plate disposed between the first and second cylinder bodies; and first and second shaft supporting members mounted on the first and second cylinder bodies so as to close the discharge openings of the first and second compressing compartments, respectively, while supporting the rotational shaft, and the first and second mufflers may be equipped to outer surfaces of the first and second shaft supporting members, respectively.

The compressor may further comprise a closed container to contain all the components described above, and a discharge pipe coupled to the closed container to discharge the gas within the closed container to the outside.

The first discharge flow path may comprise an extension pipe extended from the first muffler into an inner space of the closed container and having a predetermined length, and the second discharge flow path may penetrate through the first and second cylinder bodies, and the partition plate to communicate with the inner space of the closed container outside the first muffler.

The first and second discharge flow paths may be formed at a plurality of locations spaced apart from each other.

The first discharge flow path may comprise a discharge opening formed at a center of the first muffler such that an inner surface of the discharge opening is spaced from the outer surface of the first shaft supporting member; and a first extension pipe vertically extended a predetermined length above the discharge opening of the first muffler such that the first extension pipe surrounds the outer surface of the first shaft supporting member, with an inner surface of the first extension pipe spaced apart from the outer surface of the first shaft supporting member, and the second discharge flow path may penetrate through the first and second cylinder bodies, and the partition plate to communicate with the inner space of the closed container outside the first muffler.

The second discharge flow path may comprise a second extension pipe penetrating through the first shaft supporting member and the first muffler, and extended a predetermined length into the inner space of the closed container.

The first discharge flow path may comprise a first discharge opening formed at a center of the first muffler such that an inner surface of the discharge opening is spaced from an outer surface of the first shaft supporting member, and an extension pipe vertically extended a predetermined length above the discharge opening of the first muffler such that the extension pipe surrounds the outer surface of the first shaft supporting member, with an inner surface of the extension pipe spaced apart from the outer surface of the first shaft supporting member, and the second discharge flow path may comprise an extension extended from a periphery of the second muffler towards the first muffler so as to surround the outer surfaces of the second shaft supporting member and the second cylinder body in a state of being separated from the outer surfaces of the second shaft supporting member and the second cylinder body.

The compressor may further comprise a communication flow path formed in the first cylinder body such that the second discharge flow path communicates with the inner space of the closed container where the first muffler is installed in the closed container.

Each of the first and second compressing devices may comprise: an eccentric portion provided on an outer surface of the rotational shaft to perform the compression of gas while rotating within an associated compressing compartment; a ring piston coupled to an outer surface of the eccentric portion to allow the eccentric portion to rotate with an outer surface of the ring piston in contact with an inner surface of the associated compressing compartment; and a vane to partition an inner space of the compressing compartment while linearly traveling in a radial direction according to rotation of the ring piston.

In accordance with another aspect, a multi-cylinder compressor is provided comprising: first and second compressing compartments partitioned from each other; first and second mufflers equipped to discharge openings of the first and second compressing compartments, respectively; and a plurality of flow paths, each having a narrow and elongated shape and being extended from the discharging openings of the first and second mufflers to act as a sound filter to reduce noise and pulsation at the discharge opening of an associated muffler.

### BRIEF DESCRIPTION OF THE DRAWINGS

These and/or other aspects and advantages of the invention will become apparent and more readily appreciated from the following description of the exemplary embodiments, taken in conjunction with the accompanying drawings, in which:

FIG. 1 is a cross-sectional view illustrating the construction of a multi-cylinder compressor in accordance with a first embodiment of the present invention;

FIG. 2 is a cross-sectional view taken along line II-II′ of FIG. 1;

FIG. 3 is a cross-sectional view illustrating the construction of a multi-cylinder compressor in accordance with a second embodiment of the present invention; and

FIG. 4 is a cross-sectional view illustrating the construction of a multi-cylinder compressor in accordance with a third embodiment of the present invention.

### DETAILED DESCRIPTION OF THE ILLUSTRATIVE, NON-LIMITING EMBODIMENTS OF THE INVENTION

Reference will now be made in detail to illustrative, non-limiting embodiments of the invention, examples of which are illustrated in the accompanying drawings, wherein like reference numerals refer to the like elements throughout the drawings. The exemplary embodiments are described below to explain the invention by referring to the figures. It should be understood that, although the present invention may be applied to a reciprocation type compressor, a scroll type compressor, and a linear compressor, which have a plurality of compressing compartments, the following embodiments are described as an example using a multi-cylinder compressor, which compresses a refrigerant.

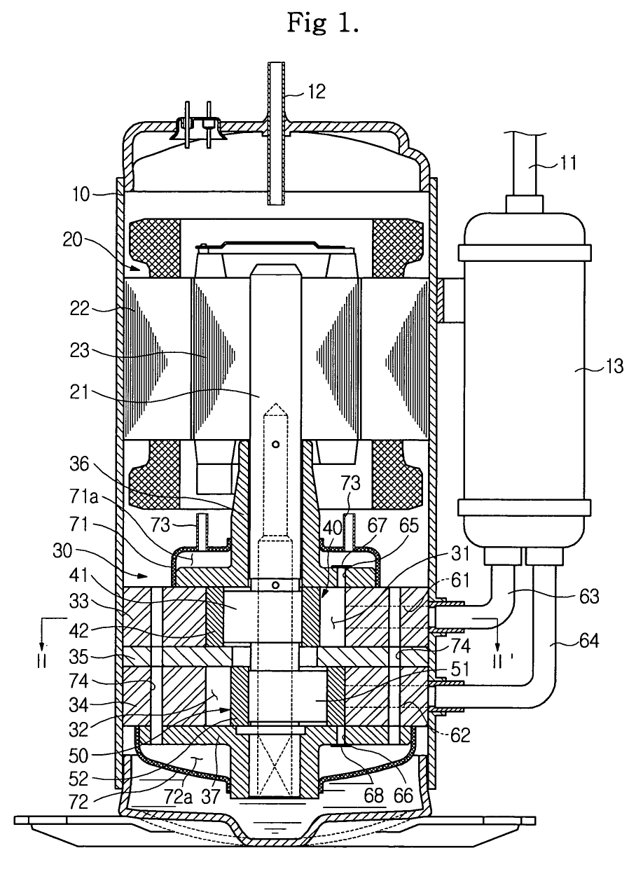

Referring to FIG. 1, a multi-cylinder compressor in accordance with a first embodiment of the present invention comprises a motor 20 disposed at an upper portion inside a closed container 10 to generate a rotational force, and a compressing part 30 disposed at a lower portion inside the closed container 10 while being connected to the motor 20 through a rotational shaft 21.

The motor 20 includes a cylindrical stator 22 fixed to an inner surface of the closed container 10, and a rotor 23 rotatably installed inside the stator 22 while being coupled at the center of the rotor 23 to the rotational shaft 21.

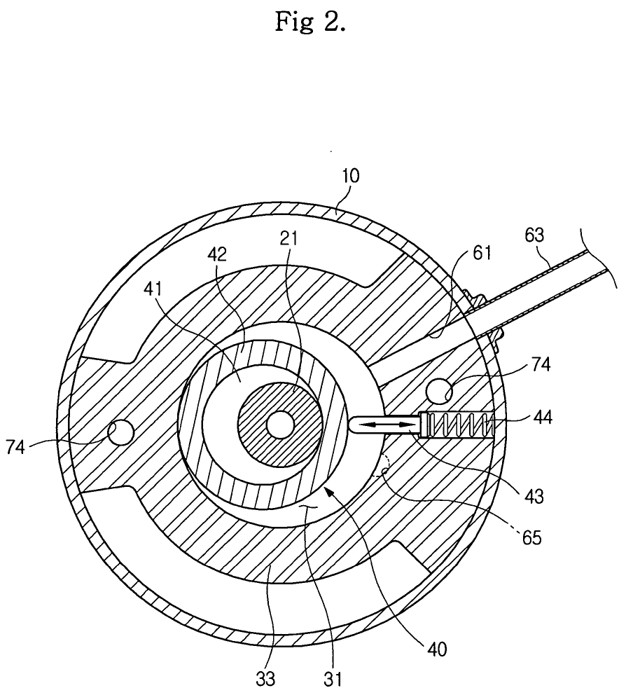

As shown in FIGS. 1 and 2, the compressing part 30 includes a first cylinder body 33 provided at an upper portion thereof and having a first cylindrical compressing compartment 31 formed in the first cylinder body 33, a second cylinder body 34 provided at a lower portion and having a second cylindrical compressing compartment 32 formed in the second cylinder body 34, and first and second compressing devices 40 and 50 installed within the first and second compressing compartments 31 and 32 to perform compression of a refrigerant, respectively. The rotational shaft 21 extended from the motor 20 is installed to penetrate through the center of the first and second compressing compartments 31 and 32 so as to operate the first and second compressing devices 40 and 50 within the first and second compressing compartments 31 and 32.

The compressing part 30 includes a partition plate 35 disposed between the first and second cylinder bodies 33 and 34 in order to partition the first compressing compartment 31 provided at the upper portion of the compressing part from the second compressing compartment 32 provided at the lower portion of the compressing part, and first and second shaft supporting members 36 and 37 mounted on an upper side of the first cylinder body 33 and a lower side of the second cylinder body 34, respectively, so as to close upper and lower openings of the first and second compressing compartments 31 and 32, respectively, while supporting the rotational shaft 21.

The first and second compressing devices 40 and 50 respectively installed within the first and second compartments 31 and 32 include first and second eccentric portions 41 and 51 provided on outer surfaces of the rotational shaft 21 in the compressing compartments 31 and 32, first and second ring pistons 42 and 52 rotatably coupled to outer surfaces of the first and second eccentric portions 41 and 51 to rotate with outer surfaces of the ring pistons 42 and 52 in contact with inner surfaces of the compressing compartments 31 and 32, first and second vanes 43 (the second vane is not shown) to partition the inner spaces of the compressing compartments 31 and 32 into an intake side and a discharge side, respectively, while linearly traveling in a radial direction within the compressing compartments 31 and 32 according to rotation of the respective ring pistons 42 and 52, and first and second vane springs 44 (the second spring is not shown) to press the vanes towards the ring pistons 42 and 52, respectively. FIG. 2 is a cross-sectional view illustrating the construction of the first compressing compartment 31, and shows the first compressing device 40, the first vane 43, and the first vane spring 44. Here, since the construction of the second compressing compartment 32 is substantially the same as that of the first compressing compartment 31, except that the second eccentric portion 51 is disposed opposite to the first eccentric portion 41, the construction of the second compressing compartment 40 and the second vane is not shown in FIG. 2.

The first and second cylinder bodies 33 and 34 have first and second intake ports 61 and 62 connected to first and second intake pipes 63 and 64, respectively, such that a refrigerant gas flows in first and second cylinder bodies 33 and 34 therethrough. The first and second supporting members 36 and 37 have first and second discharge ports 65 and 66 having first and second discharging valves 67 and 68, respectively, in order to discharge compressed refrigerant gas. In FIG. 1, reference numeral 13 denotes an accumulator installed within a refrigerant intake pipe 11, and reference numeral 12 denotes a discharge pipe to guide the compressed refrigerant inside the closed container 10 to the outside.

In such a multi-cylinder compressor, as the first and second eccentric portions 41 and 51 provided on the rotational shaft 21 in the first and second compressing compartments 31 and 32 are rotated by virtue of driving of the motor 20, the first and second ring pistons 42 and 52 intake the refrigerant gas from the first and second intake ports 61 and 62, and discharge the compressed refrigerant towards the first and second discharge ports 65 and 66 while eccentrically rotating within the first and second compressing compartment 31 and 32, respectively, thereby performing operations for compressing the refrigerant gas.

When the compression of the refrigerant gas is performed as described above, the compressed refrigerant gas discharged through the first and second discharge ports 65 and 66 is accompanied with noise and pulsation. Accordingly, the compressor of the present invention further includes first and second cup-shaped mufflers 71 and 72 installed to cover an upper portion of the first shaft supporting member 36 and a lower portion of the second shaft supporting member 37, respectively, so as to reduce the noise and pulsation of the discharge gas. That is, this construction can reduce the noise and pulsation of the discharge gas according to an interference phenomenon between the noise and pulsation of the refrigerant gas discharged from the respective discharge ports 65 and 66 and the noise and pulsation reflected within inner spaces 71a and 72a of the mufflers 71 and 72.

The first and second mufflers 71 and 72 are provided with first and second discharge flow paths 73 and 74 in order to further enhance noise and pulsation reduction of the discharge gas while guiding the discharge of the compressed gas, respectively. The first discharge flow path 73 consists of an extension pipe perpendicularly extended a predetermined length from a discharge opening, which is provided at either side through the upper surface of the first muffler 71, to the outside of the first muffler 71. The second discharge flow path 74 consists of a flow path penetrating the first and second cylinder bodies 33 and 34, the partition plate 35, and the second shaft supporting member 37 such that an inner space 72a of the second muffler 72 communicates with an inner space at an upper portion of the closed container 10 where the motor 20 is disposed in the closed container 10. At this time, the second discharge flow path 74 is also provided at either side of the compressing part.

Such a construction can further reduce the noise and pulsation of the discharge gas by not only inducing interference between an incidence wave and a reflection wave of the compressed gas passing through the inner spaces 71a and 72a of the first and second mufflers 71 and 72, but also allowing the compressed gas to pass through the narrow and elongated discharge flow paths 73 and 74. That is, in this construction, the first and second discharge flow paths 73 and 74 can act as a sound filter, and remarkably reduce noise and pulsation within a specific frequency band. As a result, the construction of the invention can provide remarkably enhanced noise and pulsation reduction in comparison to the conventional noise and pulsation reduction device in which the discharge opening of the muffler is directly communicated with the interior of the closed container 10.

Moreover, according to the present invention, since the noise and pulsation of the gas discharged into the interior of the closed container 10 is reduced as the discharge gas passes through the first and second flow paths 73 and 74, and exits of the first and second discharge flow paths 73 and 74 are separated from each other, so that the noise and pulsation of the discharge gas injected into the inner portion of the closed container 10 can be prevented from being amplified around the exits of the first and second discharge flow paths 73 and 74. That is, as for the conventional compressor, the compressed gas discharged from the respective compressing compartments is joined within the first muffler, and causes interaction of the noise and pulsation caused by the discharge gas from the respective compressing compartments therein, thereby amplifying the noise and pulsation within a specific frequency band, which can be easily transferred into the closed container, leading to unsatisfactory noise and pulsation reduction of the discharge gas.

Meanwhile, in the noise and pulsation reduction device constructed as described above, an excessively narrow width of the first and second discharge flow paths 73 and 74 results in an increased flow loss of the discharge gas. Accordingly, in order to securely provide an appropriate discharge flow path, it is desirable that the noise and pulsation be reduced by adjusting the sound resistance of the discharge flow path in a manner of extending the length of the first and second discharge flow paths 73 and 74 while enlarging the width of these flow paths 73 and 74

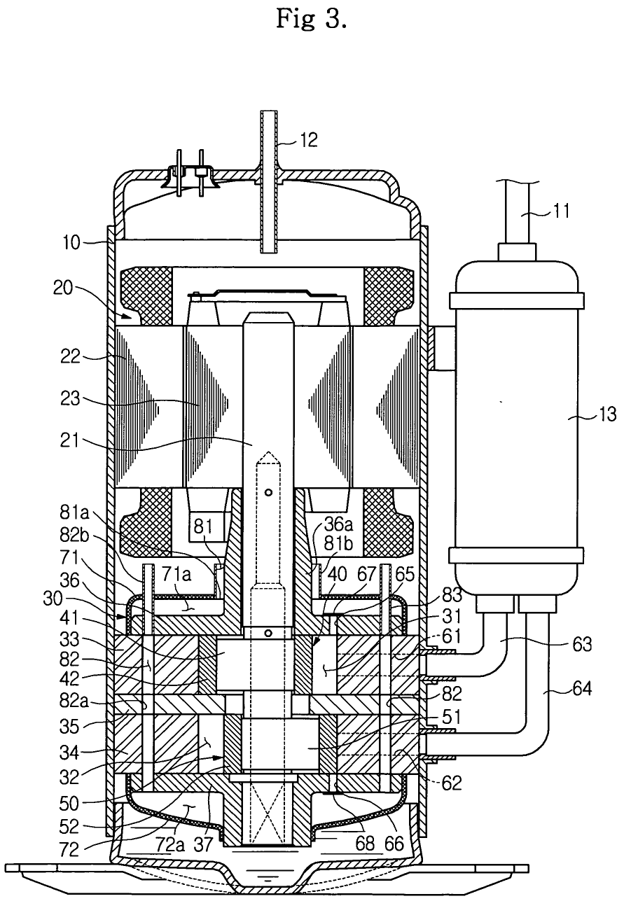

FIG. 3 shows a multi-cylinder compressor in accordance with a second embodiment of the present invention. Compared with the compressor according to the first embodiment described above, there is a difference in construction of first and second discharge flow paths 81 and 82.

The first discharge flow path 81 comprises a discharge opening 81a formed at a center of the first muffler 71, and a first extension pipe 81b vertically extended a predetermined length above the upper surface of the first muffler 71 from the first discharge opening 81a so as to surround the outer surface of the first shaft supporting member 36. At this time, the inner surface of the first extension pipe 81b is spaced from the outer surface of a shaft supporting portion 36a of the first shaft supporting member 36, thereby forming the first discharge flow path 81, which can reduce the noise and pulsation while guiding the discharge of the compressed gas. Additionally, the second discharge flow path 82 comprises a flow path 82a, which penetrates through the first and second cylinder bodies 33 and 34, the partition plate 35, and the second shaft supporting member 37, and a second extension pipe 82b extended a predetermined length into the inner space of the closed container 10 after penetrating the first shaft supporting member 36 and the first muffler 71. Although the first and second discharge flow paths 81 and 82 have some difference in construction from that of the first embodiment, the flow paths 81 and 82 have similar functions to those of the first embodiment. Accordingly, detailed description thereof will be omitted below.

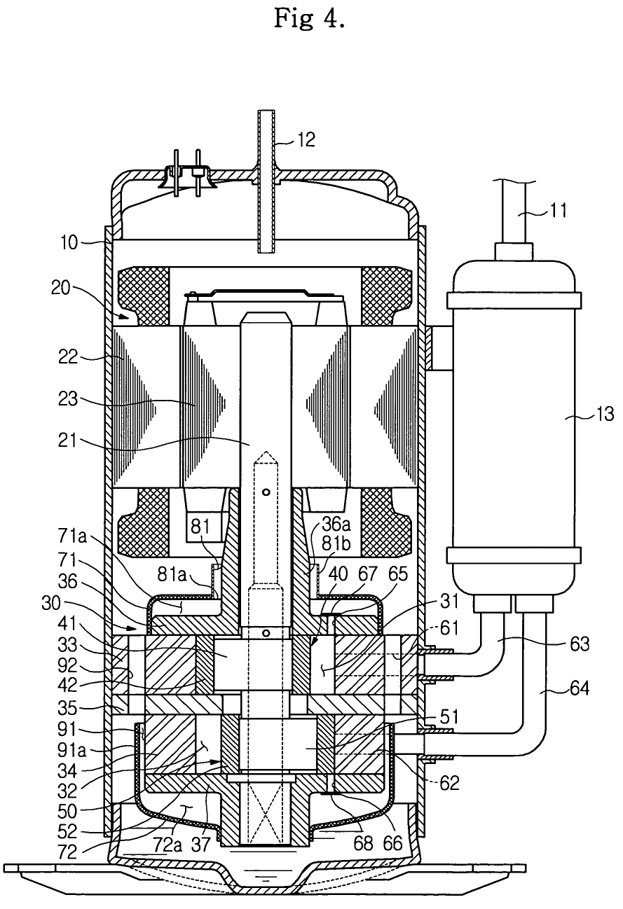

FIG. 4 shows a multi-cylinder compressor in accordance with a third embodiment of the present invention. Compared with the compressor according to the second embodiment described above, there is a difference in construction of a second discharge flow path 91. The second discharge flow path 91 is formed by means of an extension 91a extended a predetermined length from a periphery of the second muffler 72 towards the first muffler 71 so as to surround the outer surface of the second shaft supporting member 37 and the second cylinder body 34 in a state of being separated from the outer surface thereof. That is, the inner surface of the extension 91a surrounding the second muffler 72 is spaced a predetermined distance from the outer surface of the second shaft supporting member 37 and the second cylinder body 34, thereby forming the second discharge flow path 91, which can reduce the noise and pulsation while guiding the discharge of the compressed gas. For the construction described above, the cylinder body 34 has an outer diameter less than an inner diameter of the closed container 10 such that the outer surface of the second cylinder body 34 is spaced from the inner surface of the closed container 10. Additionally, according to the third embodiment, the compressor further comprises a communication flow path 92 formed through the first cylinder body 33 and the partition plate 35, such that the second discharge flow path 91 communicates with the inner space of the closed container 10 where the first muffler 71 is installed in the closed container 10. Although the third embodiment of the invention has some difference from the first and second embodiments, the flow paths 91 and 92 have similar functions to those of the first and second embodiments. Accordingly, detailed description thereof will be omitted below.

As apparent from the above description, the multi-cylinder compressor according to the present invention allows the noise and pulsation of the gas discharged from the first muffler to be reduced while the discharge gas passes through the first narrow and elongated discharge flow path, and allows the noise and pulsation of the gas discharged from the second muffler to be also reduced while the discharge gas passes through the second narrow and elongated discharge flow path, thereby remarkably reducing the noise and pulsation of the discharge gas in comparison to the conventional multi-cylinder compressor.

Moreover, the compressed gas is independently discharged from the respective compressing compartments into the inner space of the closed container through the first and second discharge flow paths, which are separated from each other, thereby preventing the amplification of the noise and pulsation.

Moreover, unlike the conventional multi-cylinder compressor, according to the present invention, the noise and pulsation reduction is further enhanced only with the first and second discharge flow paths without preparing a separate Helmholtz resonator, thereby allowing easy manufacturing of the multi-cylinder compressor compared with the conventional multi-cylinder compressor.

Although exemplary embodiments of the invention have been disclosed for illustrative purposes, those skilled in the art will appreciate that various modifications, additions and substitutions are possible, without departing from the scope and spirit of the invention as disclosed in the accompanying claims.

What is claimed is:

1. A multi-cylinder compressor, comprising: first and second compressing compartments partitioned from each other to perform compression of gas, respectively; first and second mufflers equipped to discharge openings of the first and second compressing compartments, respectively; and first and second discharge flow paths extended a predetermined length from the first and second mufflers so as to reduce noise and pulsation while guiding discharge of compressed gas.

2. The compressor according to claim 1, wherein both of the first and second discharge flow paths have a length larger than a width of a cross section thereof.

3. The compressor according to claim 1, further including: first and second cylinder bodies constituting the first and second compressing compartments, respectively; first and second compressing devices disposed within the first and second compressing compartments, respectively; a rotational shaft penetrating through the first and second compressing compartments to drive the first and second compressing devices; a partition plate disposed between the first and second cylinder bodies; and first and second shaft supporting members mounted on the first and second cylinder bodies so as to close the discharge openings of the first and second compressing compartments, respectively, while supporting the rotational shaft, wherein the first and second mufflers are equipped to outer surfaces of the first and second shaft supporting members, respectively.

4. The compressor according to claim 3, further comprising: a closed container to contain all components of the compressor, and a discharge pipe coupled to the closed container to discharge the gas within the closed container to the outside.

5. The compressor according to claim 4, wherein the first discharge flow path comprises an extension pipe extended from the first muffler into an inner space of the closed container and having a predetermined length, and the second discharge flow path penetrates through the first and second cylinder bodies, and the partition plate to communicate with the inner space of the closed container outside the first muffler.

6. The compressor according to claim 5, wherein the first and second discharge flow paths are formed at a plurality of locations spaced apart from each other.

7. The compressor according to claim 4, wherein the first discharge flow path comprises a discharge opening formed at a center of the first muffler such that an inner surface of the discharge opening is spaced from an outer surface of the first shaft supporting member, and a first extension pipe vertically extended a predetermined length above the discharge opening of the first muffler such that the first extension pipe surrounds the outer surface of the first shaft supporting member, with an inner surface of the first extension pipe spaced apart from the outer surface of the first shaft supporting member, and wherein the second discharge flow path penetrates through the first and second cylinder bodies, and the partition plate to communicate with the inner space of the closed container outside the first muffler.

8. The compressor according to claim 7, wherein the second discharge flow path comprises a second extension pipe penetrating through the first shaft supporting member and the first muffler, and extended a predetermined length into the inner space of the closed container.

9. The compressor according to claim 4, wherein the first discharge flow path comprises a discharge opening formed at a center of the first muffler such that an inner surface of the discharge opening is spaced from an outer surface of the first shaft supporting member, and an extension pipe vertically extended a predetermined length above the discharge opening of the first muffler such that the extension pipe surrounds the outer surface of the first shaft supporting member, with an inner surface of the extension pipe spaced apart from the outer surface of the first shaft supporting member, and wherein the second discharge flow path comprises an extension extended from a periphery of the second muffler towards the first muffler so as to surround the outer surfaces of the second shaft supporting member and the second cylinder body in a state of being separated from the outer surfaces of the second shaft supporting member and the second cylinder body.

10. The compressor according to claim 9, further comprising a communication flow path formed in the first cylinder body such that the second discharge flow path communicates with the inner space of the closed container where the first muffler is installed in the closed container.

11. The compressor according to claim 3, wherein each of the first and second compressing devices comprises: an eccentric portion provided on an outer surface of the rotational shaft to perform the compression of gas while rotating within an associated compressing compartment; a ring piston coupled to an outer surface of the eccentric portion to allow the eccentric portion to rotate with an outer surface of the ring piston in contact with an inner surface of the associated compressing compartment; and a vane to partition an inner space of the compressing compartment while linearly traveling in a radial direction according to rotation of the ring piston.

12. A multi-cylinder compressor, comprising: first and second compressing compartments partitioned from each other; first and second mufflers equipped to discharge openings of the first and second compressing compartments, respectively; and a plurality of flow paths, each having a narrow and elongated shape and being extended from the discharging opening of an associated muffler to act as a sound filter to reduce noise and pulsation at the discharge opening of the associated muffler.
